(19) United States
(12) Patent Application Publication
(10) Pub. No.: US 2003/0187515 A1
(43) Pub. Date: Oct. 2, 2003

(54) Collagen biofabric and methods of preparing and using the collagen biofabric

(76) Inventors: Robert J. Hariri, Florham Park, NJ (US); Aleksandr M. Kaplunovsky, Rockaway, NJ (US); Patricia A. Murphy, Wayne, NJ (US)

Correspondence Address: CRAIG G. COCHENOUR, BUCHANAN INGERSOLL, P.C., ONE OXFORD CENTRE, 20th FLOOR, 301 GRANT STREET, PITTSBURGH, PA, 15219, US

(21) Appl. No.: 10/106,653

(22) Filed: Mar. 26, 2002

(51) Int. Cl. A61F 2/02

(52) U.S. Cl. 623/23.72; 623/1.41; 623/915; 435/378; 435/399; 424/583

(57) ABSTRACT

A method of preparing a placental-derived amniotic membrane biofabric is provided. The biofabric is a dry decellularized amniotic membrane that is capable of being stored at room temperature, and subsequent to rehydration can be used for a variety of medical and/or research purposes. A laminate of said biofabric is also provided that can be shaped into complex shapes and repopulated with cells to generate both acellular and cellularized engineered tissues and organoids.

### BACKGROUND OF THE INVENTION

1. Field of the Invention

The present invention is generally in the area of cell-free human placental-derived amniotic membranes for use in tissue graft surgical procedures. Because the present amniotic membrane is never frozen during any step in its preparation and storage, it has improved tensile strength and suturability properties resulting in reduced preparation time in the surgical room by eliminating thawing time. The present improved amniotic membrane is termed “biofabric”. Activation of the improved biofabric is simplified since no backing or support are required for storage and handling reducing activation time in solution. Furthermore, the improved biofabric is cell-free which reduces immunogenicity and graft rejection by the host and improves uniformity, smoothness, and clarity required in grafts of the eye.

2. Description of the Background Art

The scarcity of human donor tissues for grafting is a growing problem that has stimulated the development of new materials for tissue grafting. Most often these sources of biological raw material are scarce, difficult to obtain, and very costly. Possible potential problems with xenogenic tissues (tissues from other species) carrying zoonotic diseases or causing cross-species rejection have made these tissues less desirable.

Allogenic grafts, or grafts from different individuals of the same species, continue to be the preferred source for human graft materials. Human placental membranes comprise the elastic, water permeable sac that houses the developing fetus and amniotic fluid. The membranes are constructed of two (2) laminated layers composed of the amnion and the chorion. The amnion is constructed of a densely packed layer of collagen fibrils forming a tight beta-pleated sheet. The sheet has desirable biomechanical characteristics useful in tissue graft applications. Thus, amniotic membranes are a good source of allogenic graft material.

Amniotic membranes are disclosed in the art which have several disadvantages over the present invention. Amniotic membranes derived from human placental amnion have been described since as early as 1910. Various preparations of amniotic membranes have included preservation by saline and antibiotic mixtures, and by alcohol dehydration, with or without separation of the amnion layer from the chorion layer.

More recently, methods have been disclosed which rely on freezing for preservation of the amniotic membrane for application in tissue graft surgical procedures to correct corneal epithelial defects. See U.S. Pat. Nos. 6,152,142 and 6,326,019B1 (Tseng). Tseng discloses an amniotic membrane that is mounted on a substrate and preserved in a mixture of Dulbecco's Modified Eagle Medium and glycerol and frozen at −80° C.

The process of freezing the tissue at any time during its preparation makes the Tseng amniotic membrane brittle, and even more brittle after the steps of thawing and activation. In addition, the thawing and activation steps add time required for the handling of the amniotic membrane. Furthermore, because of the brittleness of the Tseng amniotic membrane caused by the freezing step in the preservation and preparation process, a structural support or backing is required to ensure structural integrity of the Tseng amniotic membrane during storage. This presents the added difficulty of separating the preserved amniotic membrane from the backing, which due to its brittleness can be difficult to handle and separate intact. The added manipulation required for the separation of the amnion membrane from the backing increases the likelihood of rupture of the membrane, leading to further increased preparation time in the surgical suite prior to performing the tissue graft surgery The presence of the backing as a structural support also increases the length of time required to activate the amniotic membrane to allow for thorough impregnation of the activation solution into the frozen amniotic membrane prior to performing the surgical procedure. Storage and shipping are also complicated by the requirement of −80° C. freezing.

In spite of this background art, there remains a very real and substantial unmet need for an amniotic membrane material that is easily stored and shipped without low temperature cryopreservation or freezing, requires minimal handling and activation procedures prior to grafting, and can be stored at room temperature for long periods of time and maintains superior tensile strength, superior suturability, and low immunogenicity resulting in reduced incidence of host-graft rejection, and a method of producing the same.

### SUMMARY OF THE INVENTION

The present invention has met the above-described need. The present invention provides a method for preparing placental membranes including separating the amnion and chorion layers from each other, removing the remaining cellular constituents and debris from the amnion layer while preserving the underlying extracellular matrix architecture, washing the decellularized amnion layer, and heat-drying the decellularized membrane under vacuum. This method yields a dehydrated, acellular biofabric that can remain stable under sterile storage conditions at room temperature and that is subsequently rehydrated and grafted to or implanted into a patient.

The present invention provides a placental-derived amniotic membrane or biofabric having superior characteristics of increased tensile strength, suturability, and reduced immunogenicity resulting in reduced host-graft rejection. The present invention provides a placental-derived amniotic membrane or biofabric that can be stored as dehydrated sheets without freezing or cryopreservation. Preferably, the placental-derived amniotic membrane is derived from a human placenta for use in human patients. However, the same methods can be employed using placentas from various animal species for veterinary use in animal patients.

The present invention provides a biofabric that can be used as a single layer dressing for wounds, burns, to assist post-surgical healing as a corneal or skin tissue graft, as well as a circumferential covering over the anastomotic sites of blood vessels (or vessels-to-grafts) during vascular surgery procedures to prevent leakage of blood from the suture lines and prevent the body from forming adhesions to the suture material. Similarly, this biofabric can be used as a covering over the anastomotic sites of the gastrointestinal tract during GI surgery to prevent leakage of intestinal fluids and bile from the suture lines and prevent the body from forming adhesions to the suture material. The amniotic membrane can also be laminated into multi-layer sheets or assembled into complex three-dimensional structures from laminates and/or other configurations that can be populated with living cells in discrete and structured designs to generate both cellular and cellularized engineered tissues and or organoids.

A biofabric is described that is prepared by the process described herein and that can be used for a variety of medical purposes.

### BRIEF DESCRIPTION OF THE DRAWINGS

FIG. 1 shows the chorion and amniotic membranes of a human placenta.

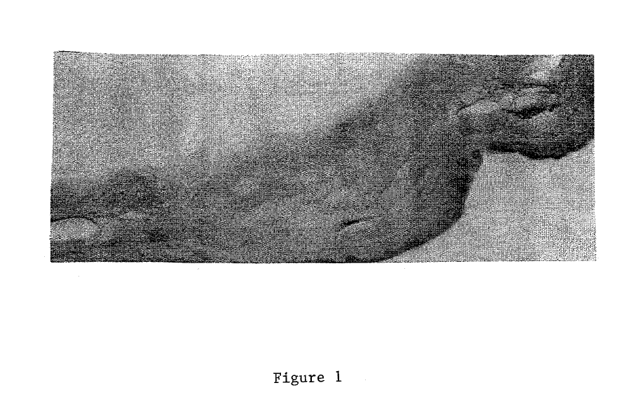

FIG. 2 shows the beta-pleated sheet structure of the amnion membrane formed by densely packed layer of collagen fibrils.

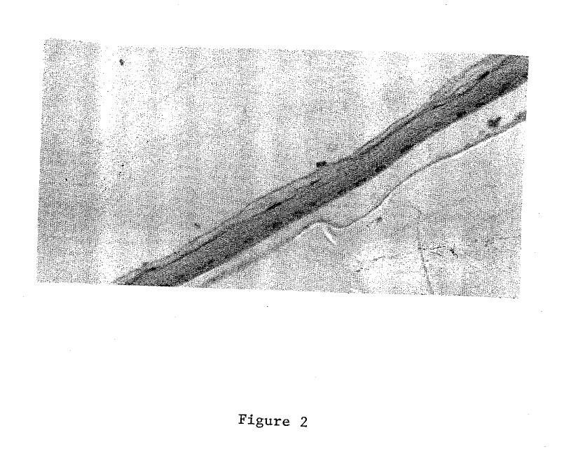

FIG. 3 shows the mesh frame and biofabric (tissue) being dried therein.

FIG. 4 shows the biofabric having a uniform translucent surface with an embossed pattern.

FIG. 5 shows a sectional view of the biofabric having differential fiber compression resulting in increased tensile strength.

### DETAILED DESCRIPTION OF THE INVENTION

As used herein, the term “patient” includes members of the animal kingdom including for example but not limited to human beings.

I. Method of Producing the Biofabric

Following normal birth, the placenta, umbilical cord and umbilical cord blood are spontaneously expelled from the contracting uterus. The expectant mother is screened at the time of birth for communicable diseases such as HIV, HBV, HCV, HTLV, Syphilis, CMV and other viral, bacterial, and other pathogens that could contaminate the placental tissues being collected. Only tissues collected from donors whose mothers tested negative or non-reactive to the above-mentioned pathogens are used to produce this biofabric for transplantation.

Step I

The placenta, umbilical cord, and umbilical cord blood are collected following birth. The materials are transported to the laboratory where they are processed under aseptic conditions in a clean room having a HEPA filtration system. The umbilical cord is separated from the placental disc. Prior to cutting the placental membrane and starting from the edge of the placental membrane, the amniotic membrane is separated from the chorion membrane using blunt dissection with gloved fingers. Following separation of the amniotic membrane from the chorionic membrane and placental disc, the umbilical cord stump is cut with scissors and detached from the placental disc. If separation of the anmion and chorionic membranes is not possible without tearing the tissue, the amnion and chorionic membranes can be cut from the placental disc as one piece and then peeled apart. Once separated, the chorionic membrane can be collected and saved for other uses or discarded.

The amniotic membrane is then placed in a sterile stainless steel tray filled with 0.9% NaCl solution. The amniotic membrane can be stored refrigerated at 4° C. (Centigrade) in the 0.9% NaCl solution. Preferably, the separated amniotic membrane can be stored under refrigeration for a maximum of 72 hours from the time of delivery prior to the next step in the process.

Step II

The amniotic membrane is then transferred into a clean sterile stainless steel tray, rinsed with sterile water and dried with sterile gauze. The amnion is then placed on a sterile tray with the maternal side facing upward. Using a sterile Cell Scraper (32 cm, PE blade, PS handle, NalgeNunc International), all visible cellular material is removed from the maternal side of the amniotic membrane. Sterile water is used to assist in the removal of cells and cellular debris, if needed

After completing the partial decellularization on the maternal side of the amniotic membrane, the amniotic membrane is then turned over so that the fetal side is now facing up. All visible cellular debris is gently removed with a Cell Scraper using minimal pressure on the amniotic membrane to prevent tearing. Sterile water may be used to assist in the removal of the cells and debris.

Step III

Next, the partially decellularized amniotic membrane is placed into a sterile container which is then filled with a decellularizing solution (hereinafter “D-Cell”) in an amount to cover the amniotic membrane. The decellularizing solution, such as for example but not limited to, is made of 0.1-1.0% deoxycholic acid sodium salt monohydrate (M.W.=432.59, ICN Biomedicals Inc., 1263 South Chillicotle Road, Aurora, Ohio 44202) in sterile water. As will be appreciated by those persons skilled in the art, any suitable decellularizing solution may be employed. The container with the amniotic membrane and D-Cell solution is then sealed and placed on a rocking platform (Model 100, VWR Scientific Products Corp., P.O. Box 640169, Pittsburgh, Pa. 15264-0169). The amniotic membrane in the D-cell solution is then agitated for at least 15 minutes on the rocking platform. After the agitation step, the amniotic membrane is removed from the container and placed in a clean sterile stainless steel tray filled with sterile 0.9% NaCl solution.

Using a new sterile Cell Scraper, residual D-cell solution is removed and any remaining cellular material is removed form both sides of the amniotic membrane. This step may be repeated as many times as necessary to remove all visible residual cellular material from both sides of the amniotic membrane. The decellularized amniotic membrane may be stored in sterile 0.9% NaCl solution prior to proceeding to Step IV set forth below.

Step IV

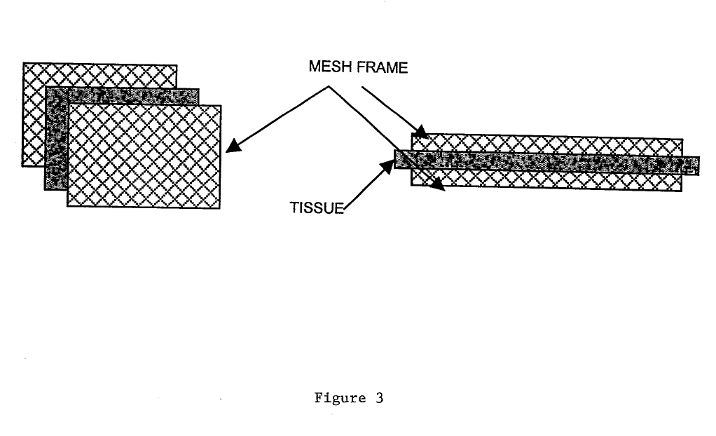

Remove the decellularized amniotic membrane from the 0.9% NaCl solution and gently squeeze out the excess fluid. The decellularized amniotic membrane is then gently stretched until it is flat with the fetal side faced in a downward position on the tray. Flip the decellularized amniotic membrane over and place it on a plastic mesh drying frame (Quick Count® Plastic Canvas, Uniek, Inc., Waunakee, Wis.) with about 0.5 centimeter (cm) of amniotic membrane overlapping the edges of the drying frame. The fetal side should be facing upwards. The overlapping amniotic membrane extending beyond the drying frame may be wrapped over the top of the frame with clamps or hemostats. Sterile gauze is placed on the drying platform of a heat dryer (Model 543 or 583, Bio-Rad Laboratories, 200 Alfred Nobel Drive, Hercules, Calif. 94547), covering an area slightly larger than the amniotic membrane resting on the plastic mesh drying frame. The plastic mesh drying frame is placed on top of the gauze on the drying platform so that the edges of the plastic frame extend above 0.1-1.0 cm beyond the gauze edges. Preferably, the drying frame having the amniotic membrane is placed on top of the sterile gauze with the fetal side of the amniotic membrane facing upward. Another plastic framing mesh may be placed on top of the amniotic membrane, but this is not necessary. FIG. 3 shows the amniotic membrane placed between the two mesh drying frames. Generally, just a sheet of thin plastic (SW 182, clear PVC, AEP Industries Inc., South Hackensack, N.J. 07606) is placed on top of the membrane covered plastic mesh so that it extends well beyond all of the edges and the second mesh frame is not needed. Additionally, an alternate to the above-described procedure would include deleting the plastic mesh frame entirely and placing the amniotic membrane on several sheets of Tyvek material (single layer sheets of Tyvek for medical packaging, Dupont Tyvek®, P.O. Box 80705, Wilmington, Del. 19880-0705) with one sheet of Tyvek on top of the membrane (prior to placing the plastic film). This alternate process will produce a smoother version of the same product. The dryer is turned on so that temperature of the drying platform is maintained at a low heat setting, such as for example, from about 45 to 50° C., under vacuum. Preferably, the vacuum pressure is set to about −22 inches of Hg. The amniotic membrane that is placed with the one or two mesh drying frames is heat-vacuum dried for approximately 60 minutes to achieve a dehydrated amniotic membrane. The low heat setting along with vacuum pressure allows the membrane to achieve the dehydrated state without denaturing the collagen.

After completion of the drying process, the heat dryer is opened and the amniotic membrane is cooled down for approximately two minutes with the vacuum pump running.

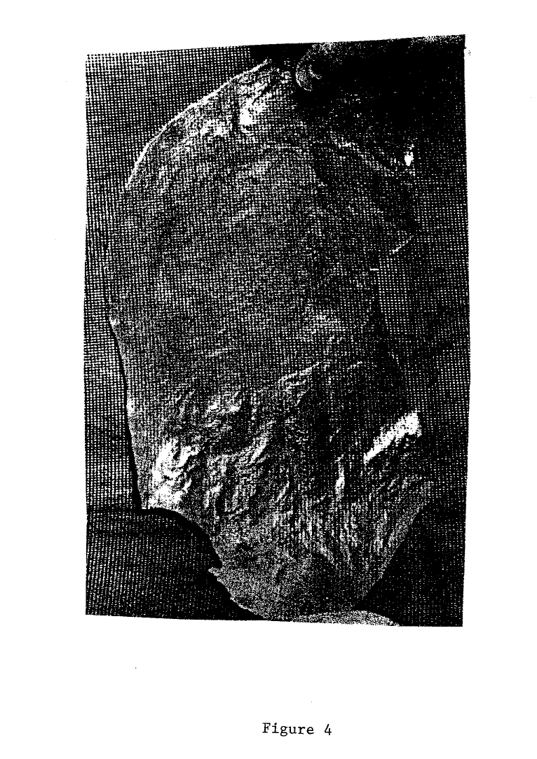

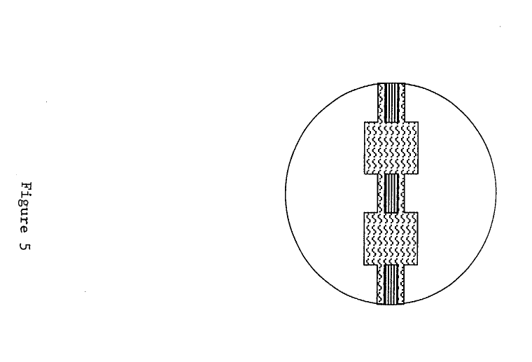

The dehydrated decellularized amniotic membrane now has its final form of a uniform, translucent biofabric (FIG. 4) made of a beta-pleated sheet cell-free matrix that has the surface pattern shown in FIG. 5. FIG. 5 shows the biofabric surface has a pattern of differential fiber compression regions along and perpendicular to the axis of the material which contributes to its superior tensile strength and suturability properties. The alternate method for producing the membrane will produce an amniotic membrane product that is smoother (without the pattern of differential fiber compression regions along and perpendicular to the axis of the material) which may be advantageous for other applications, such as enhanced cell growth when used as a matrix to expand cells. The amniotic membrane is gently lifted off the drying frame by peeling it off slowly and is placed in a sterile container (e.g. peel pouch), which is then sealed. The process of the invention enables the biofabric material to be stored in the sealed sterile containers at room temperature for 12 months or longer without any degradation.

II. Method of Using the Biofabric

The present invention will be further understood by reference to the following examples.

### EXAMPLE 1

Tissue Grafts

Various biofabric tissue samples of the present invention have been evaluated by surgeons by performing tissue grafts on pig eye specimens to determine surgical handling properties and suturability of the biofabric of the present invention.

The procedure is as follows:

1. Cut dry biofabric of the present invention to fit a single quadrant of a pig's eye.

2. Place the cut biofabric of the present invention on the surface of the pig's eye.

3. Hydrate the biofabric of the present invention with buffered sterile saline and allow graft to activate on the pig's eye. Hydration times of 2, 5, 10, and 20 minutes, respectively, were employed.

4. Suture the activated hydrated biofabric to the epithelium of the pig's eye with several 9-0 vicryl suture bites.

Results indicate that all surgeons opined that the biofabric of the present invention had superior performance when compared to amnion alternatives such as the fresh frozen membranes disclosed by Tseng, in the areas of manipulation, preparation and suturability. The biofabric performed consistently from batch to batch when compared to the amnion alternatives known in the art. The biofabric of the present invention was stronger and easier to handle than paper-backed (supported) amniotic membrane known in the art and did not cheese-wire when sutured and did not tear like fresh frozen amniotic membranes that are known in the art. The biofabric of the present invention could be sutured effectively with a range of micro-sutures form 8-0 to 10-0. The initial appearance of the pattern of the biofabric was imperceptible after 30 minutes post-suture in situ. All of the biofabric samples showed good manipulability at 2, 5, 10 and 20 minutes of hydration, ideally between 5 and 10 minutes of hydration activation.

Table I sets forth a comparison between the amniotic membrane of the present invention in comparison to the cryo-preserved amniotic membrane of U.S. Pat. No. 6,326,019 B1.

TABLE I

| | | |
|---|---|---|
| | Heat-Dried Amniotic | Frozen Amniotic |
| | Membrane Allograft | Membrane Allograft |
| | Of the Present Invention | U.S. Patent No. |
| | | 6,326,019 B1 |
| FORM | Thin, dry sheets | Frozen in solution |
| | (dehydrated) - Note: | |
| | tissue is NEVER frozen | |
| | during any step of the | |
| | process | |
| SUBSTRATE | None | Mounted on a support |
| (support) | | filter paper which must |
| | | be removed prior to use |
| STORAGE | Room temperature | Frozen in solution |
| ACTIVATION/ | Ready to use | Requires thawing time of |
| USE | immediately in its dry | 20 minutes and removal |
| | form and will activate | from the substrate |
| | (rehydrate) after only 5 | support prior to use |
| | minutes following the | |
| | addition of saline | |
| CELLULARITY | No cells | Dead cells present- |
| | (Decellularized)- | the process of freezing |
| | cells are lysed using a | and thawing the tissue |
| | detergent solution, rinsed | kills the cells on the |
| | many times and manually | membrane (however, the |
| | removed for a uniformly | cellular debris remains on |
| | thin, smooth, clear | the product) |
| | appearance | |

### EXAMPLE 2

Three-dimensional Tissue Scaffolding

In another embodiment, the biofabric of the instant invention can be assembled into laminates by layering multiple amniotic membranes into a laminate. The laminates have increased structural rigidity that allows the laminates to be shaped into complex three-dimensional structures. The shaped laminates can be populated with living cells or progenitor stem cells, wherein the stem cells may be totipotent and pluripotent stem cells, or differentiated tissue cells, in discrete and structured designs for the purpose of generating both acellular and cellularized engineered tissues or organoids.

While the above examples employ amniotic membranes derived from human placentas, it will be appreciated that both human and veterinary (animal) placentas may be employed to prepare novel amniotic membranes according to the methods of the present invention. Further, it will be appreciated by those persons skilled in the art that the amniotic membranes prepared by the method of the present invention may be used in various medical procedures, such as for example, but not limited to, as autografts and/or allografts for patients requiring such as for example, but not limited to, a surgical graft for dressing a skin wound caused by, for example, a burn or trauma, for preventing adhesions in surgery, for reconstructing mucosal surfaces, for reducing scar tissue, for reconstructing soft tissue, and for culturing many different types of cells. The biofabric of the present invention may be used to grow different cell types, such as for example endothelial cells and muscle cells, in specific regions of the biofabric. It will be appreciated by those skilled in the art that the biofabric of the present invention can be used to form, tissues and organoids, such as for example blood vessels, liver, pancreas.

Whereas particular embodiments of this invention have been described herein for purposes of illustration, it will be evident to those persons skilled in the art that numerous variations of the details of the present invention may be made without departing from the invention as defined in the appended claims.

What is claimed is:

1. A method of preparing a placental-derived amniotic membrane biofabric, comprising the steps of:
providing a placenta having placental membranes including a chorionic membrane, and an amniotic membrane;
separating the amniotic membrane from the chorionic membrane;
decellularizing the amniotic membrane to remove cells and cellular debris from the amniotic membrane;
washing the decellularized amniotic membrane to ensure removal of visible residual cellular debris; and
drying the decellularized amniotic membrane under vacuum.

2. The method of claim 1 wherein the decellularization and removal of cell debris is performed on both sides of the amniotic layer by physical scraping.

3. The method of claim 2 wherein the decellularization is performed by scraping with a cell scraper.

4. The method of claim 3 wherein the scraping is performed in sterile water or sterile saline solution.

5. The method of claim 1 wherein the washing is performed with sterile saline solution or sterile water.

6. The method of claim 4 wherein the sterile saline solution is 0.9% NaCl solution.

7. The method of claim 1 wherein the washing is performed by agitating the amniotic membrane in a decellularizing solution followed by additional decellularization by physical scraping.

8. The method of claim 1 including storing said decellularized membrane in a dry state at room temperature.

9. The method of claim 1 wherein the heat drying is performed at up to and including about 50° C. and up to and including about −22 in. of Hg vacuum pressure.

10. The method of claim 1 wherein the drying is performed in up to and including about 60 minutes.

11. The method of claim 1 wherein the placenta is a human placenta.

12. The method of claim 1 wherein the washing is performed in sterile saline solution or sterile water with agitation.

13. The method of claim 1 including placing said dry decellularized amniotic membrane to a surgical site of a patient.

14. The method of claim 13 including hydrating said dry decellularized amnion membrane at said surgical site for use as a graft.

15. The method of claim 14 including securing said hydrated decellularized amniotic membrane to said surgical site.

16. The method of claim 14 including wherein said surgical site is a patient's eye.

17. The method of claim 14 including wherein said surgical site is an internal area of the patient's body.

18. The method of claim 14 including wherein said surgical site is an external area of the patient's body.

19. The method of claim 14 including wherein said surgical site is the patient's skin.

20. A biofabric comprising a dry decellularized amniotic membrane which is stable at and is capable of being stored at room temperature.

21. The biofabric of claim 20 including epithelial cells arranged in a uniform and confluent manner.

22. A biofabric prepared by a method comprising:
providing a placenta having a chorionic membrane and an amniotic membrane;
separating said amniotic membrane from said chorionic membrane;
decellularizing said amniotic membrane to remove cells and cellular debris from said amniotic membrane;
washing said decellularized amniotic membrane to ensure removal of visible residual cellular debris; and
drying said decellularized amniotic membrane under vacuum to produce said biofabric, wherein said biofabric is capable of being stored at room temperatures.

23. The biofabric of claim 22 wherein said biofabric can be used as a single layer dressing for wounds.

24. The biofabric of claim 22 wherein said biofabric can be applied to a surgical site of a patient as a graft.

25. The biofabric of claim 22 wherein said biofabric can be applied to an internal site of a patient's body.

26. An amniotic membrane laminate prepared by a method comprising:
providing a placenta having a chorionic and an amniotic membrane;
separating said amniotic membrane from said chorionic membrane;
decellularizing said amniotic membrane to remove cells and cellular debris from said amniotic membrane;
washing said decellularized amniotic membrane to ensure removal of visible residual cellular debris;
layering at least two of said decellularized amniotic membranes in contact with each other to form said amniotic membrane laminate; and
drying said decellularized amniotic membrane laminate under vacuum to produce a dry decellularized amniotic membrane laminate that is capable of being stored at room temperature.

27. The method of claim 26 further including assembling at least one of said amniotic membrane laminates into a complex three-dimensional structure scaffold.

28. The method of claim 26 further including populating said laminates with living cells.

29. A method of producing engineered tissues or organoids, comprising:
providing a placenta having placental membranes including a chorionic membrane and an amniotic membrane;
separating the amniotic membrane from the chorionic membrane;
decellularizing the amniotic membrane to remove cells and cellular debris from the amniotic membrane;
washing the decellularized amniotic membrane to remove visible residual cellular debris;
layering at least two amniotic membranes in contact with each other to form a multi-layer amniotic membrane laminate;
shaping said laminate to form a three-dimensional structure scaffold; and
drying said decellularized amniotic membrane structure under vacuum to produce a dry decellularized amniotic membrane structure that is capable of being stored at room temperature.

30. The method of claim 29 further comprising populating said three-dimensional structure scaffold with living cells.

31. The method of claim 30 including wherein said living cells are adult tissue cells.

32. The method of claim 30 including wherein said living cells are stem cells.

33. The method of claim 32 including wherein the stems cells are totipotent.

34. The method of claim 32 including wherein the stems cells are pluripotent.

35. The method of claim 32 including wherein the stems cells are tissue specific.

36. A three-dimensional amniotic membrane laminate prepared by the method of claim 29.

37. A blood vessel prepared by the method of claim 29.
